(19) United States
(12) Patent Application Publication
(10) Pub. No.: US 2025/0246823 A1
(43) Pub. Date: Jul. 31, 2025

(54) ELECTRICAL CONNECTION DEVICE, AND ASSOCIATED ELECTRICAL UNIT AND DISTRIBUTION BOARD

(71) Applicant: Schneider Electric Industries SAS, Rueil-Malmaison (FR)

(72) Inventors: Abderrahmane Agnaou, Grenoble (FR); Louis Linares, La Terrasse (FR)

(73) Assignee: Schneider Electric Industries SAS, Rueil-Malmaison (FR)

(21) Appl. No.: 19/039,054

(22) Filed: Jan. 28, 2025

(30) Foreign Application Priority Data
Jan. 30, 2024 (FR) 2400884

(51) Int. Cl. H01R 9/24; H01R 13/22; H01R 13/621; H02B 1/04; H02B 1/56

(52) U.S. Cl. CPC H01R 9/2416; H01R 13/22; H01R 13/621; H02B 1/04; H02B 1/56

(57) ABSTRACT

This invention relates to an electrical connection device between an electrically conductive member (4A) and a terminal block (6) of an electrical unit (3). The connection device comprises a nut (23A), adapted to receive a screw (21A) which, by screwing, clamps together the electrically conductive member and the terminal block, pressing them against the nut, a core (25A), which is thermally conductive and which is bonded to the nut so as to transfer heat by conduction between the nut and the core, and a support (27A) which supports the nut and the core so as both to hold the nut securely in position and to transfer heat by conduction between the core and the support. The support is made of a material that emits a release containing microparticles and/or at least one gaseous compound when that material is heated above a predetermined temperature threshold.

This invention relates to an electrical connection device between an electrically conductive member and a terminal block of an electrical unit. It further relates to an electrical unit comprising such a connection device, and a distribution board comprising such an electrical unit.

When it comes to the electrical power units in an electrical installation, electrical units are generally mounted on a distribution board, itself housed in an electrical box or cabinet. Each electrical unit generally comprises one or more terminal blocks, to each of which electrical conductors, such as cables or busbars, are connected. Each electrically conductive member is connected and fixed at one end to one of the terminal blocks by means of a screw tightened in a nut. In the current state of the art, this nut is supported and held in position by a plastic support. This support is affixed to the housing of the electrical unit. When fitted, the screw passes through the electrically conductive member and the terminal block and is screwed into the nut, pressing the electrically conductive member against the terminal block to ensure connection. If the connection between the electrically conductive member and the terminal block is incorrectly tightened, the electrical resistance of the connection increases, and when a current flows through the connection, localised heating occurs at the connection, which is a potential source of malfunctions, or even an accident if the heating exceeds a certain threshold. For regulatory and/or safety reasons, it is therefore necessary to monitor the quality of electrical connections, in particular by monitoring the temperature rise of these connections.

It is known to install temperature sensors in the distribution board for continuous monitoring of electrical connections. However, these sensors, their installation, and the use of their measurements are expensive.

For example, JP2002081998A describes detection devices using an optical fibre installed close to electrical connections and a control unit transmitting light to the optical fibre. An abnormal temperature is detected by the loss of light transmission in the optical fibre.

It is also known to take thermal camera images during inspection visits, in order to detect hot spots on a distribution board. The only problem is that the distribution board has to be open to take the images, and for safety reasons the distribution board does not operate at full power, so the images do not accurately reflect the normal operation of the electrical units. On the other hand, opening the distribution board generates air movements that tend to cool the electrical units mounted on the panel.

U.S. Pat. No. 7,712,431B2 also describes detection devices using thermochromatic elements that change colour according to their temperature.

Another approach is to use a detection device capable of detecting gases, microparticles and volatile organic compounds, as described in EP3512056B1. In practice, a gaseous or particulate release that is abnormally emitted by unspecified electrical equipment is detected by a sensor in the detection device, which then activates an alarm.

The aim of the present invention is to provide a connection device which, while being practical to use, can effectively signal a bad connection.

To this end, the object of the invention is an electrical connection device between an electrically conductive member and a terminal block of an electrical unit, the connection device comprising:
a nut, thermally conductive and provided with first and second faces opposite each other along an axis, the first and second faces being connected to each other by an internal thread of the nut, which extends along the axis and which is adapted to receive a screw which, by screwing into the internal thread, clamps together the electrically conductive member and the terminal block, by pressing them against the first face of the nut,
a core, which is thermally conductive and is bonded to the second face of the nut so as to transfer heat by conduction between the nut and the core, and
a support, which is adapted to be affixed to an electrically insulating housing of the electrical unit and which supports the nut and the core so as both to hold the nut securely in position and to transfer heat by conduction between the core and the support, the support being made of a material which emits a release containing microparticles and/or at least one gaseous compound when this material is heated above a predetermined temperature threshold.

Thanks to the invention, when a connection between an electrically conductive member and a terminal block is incorrectly tightened, heating occurs and heats the support, which then emits a release containing microparticles and/or at least one gaseous compound. This heating is generated by the resistance, induced by the air in the space between the electrically conductive member and the terminal block due to the incorrect tightening, when a current flows through the connection between the electrically conductive member and the terminal block. In the connection device in accordance with the invention, the heat is transmitted by thermal conduction from the nut to the core, then the core transmits this heat to the support by thermal conduction via a large thermal interface represented by their surface in contact, in particular when the core is advantageously enveloped by the support, in particular by a material which emits detectable gases in the event of overheating. A gas and particle sensor at the top of the distribution board can detect the release of heat without the distribution board needing to be opened. It's also interesting to note that the release happens immediately in the event of a malfunction. This is because heat conduction is an immediate physical effect in the presence of good thermal conductors, such as the nut and core of the connection device. Any heating at the connection is immediately transmitted to the substrate, which releases gas.

In other beneficial aspects of the invention, the connection device according the invention comprises one or more of the following features, taken in isolation or in any technically possible combination:
the first face of the nut is flat and extends perpendicular to the axis.
the nut and the core are two separate parts, and the core is provided with an axial end face, which extends transversely to the axis and which is arranged in axial contact against the second face of the nut.
the second face of the nut and the axial end face of the core are flat and extend perpendicular to the axis.
the core is electrically insulating, in particular made of ceramic.
the core is provided with a free end, which is axially opposite the axial end face and which emerges from the support.
the core is integral with the nut from the second face of the nut, and the nut and core are electrically conductive, in particular made of metal.
the core is provided with a side face, which is generally cylindrical and substantially centred on the axis, and which is at least partially covered in contact by the support.

The invention also relates to an electrical unit, for example a switching device, comprising:
an electrically insulating housing,
at least one terminal block, which forms an electrical input or an electrical output of the electrical unit, and
for the or each terminal block, a connection device, as defined above, between an electrically conductive member and the terminal block.

The invention also relates to an distribution board comprising:
an electrical unit as defined above, and
for the or each terminal block, an electrically conductive member which is connected to the terminal block by the connection device.

The invention also relates to a distribution board comprising:
an electrical unit comprising:
   an electrically insulating housing,
   at least one terminal block, which forms an electrical input or an electrical output of the electrical unit, and
   for the or each terminal block, an electrically conductive member which is connected to the terminal block by the connection device, as defined above, provided with a free end projecting from the support, and
a plate to which the electrical unit is attached, the plate being thermally conductive and being in contact with the free end of the core of the connection device so as to transfer heat by thermal conduction between the core and the plate.

The invention will appear more clearly when reading the description that follows, given solely as a non-limiting example and made in reference to drawings in which:

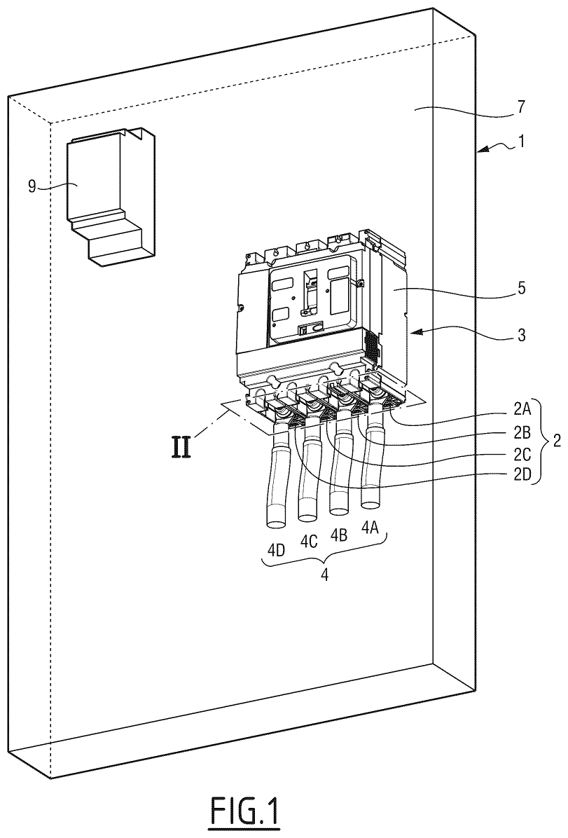

FIG. 1 is a perspective schematic view of a distribution board according to the invention;

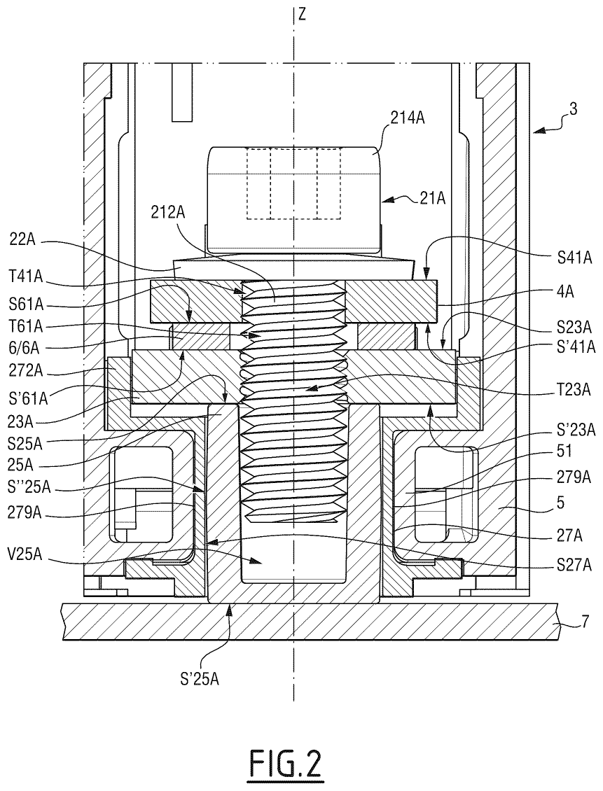

FIG. 2 shows a partial cross-section of FIG. 1, along plane II of FIG. 1;

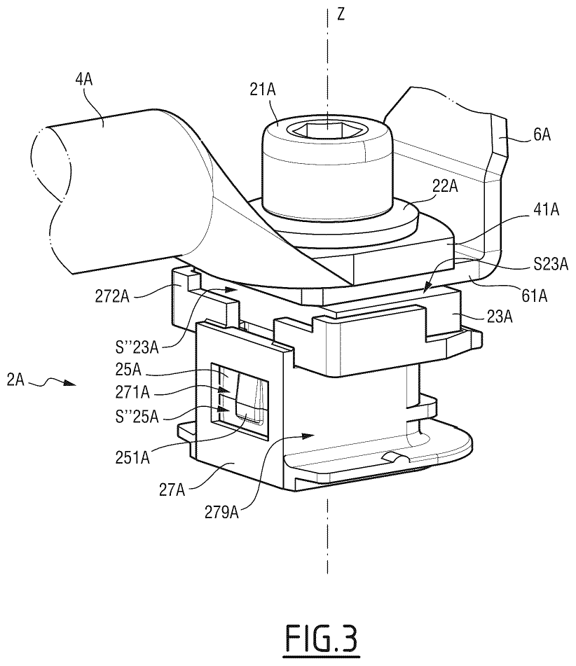

FIG. 3 is a perspective view of part of the distribution board of FIG. 1;

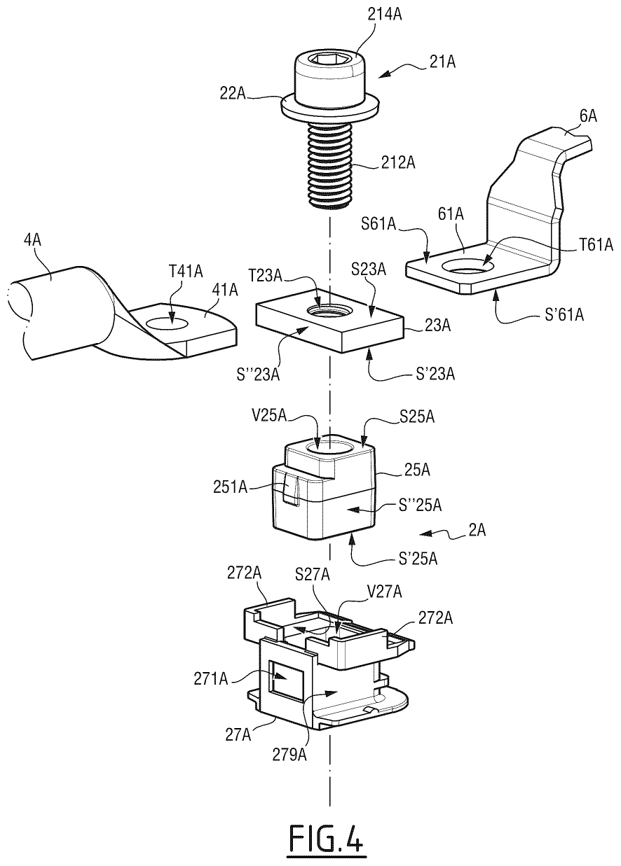

FIG. 4 is an exploded view of the components shown in FIG. 3; and

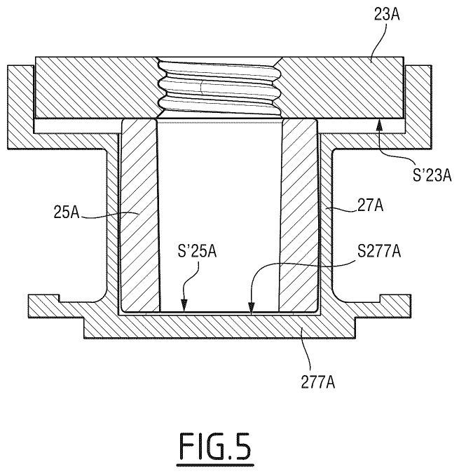

FIG. 5 is a similar view to the one in FIG. 2, for an alternative embodiment.

A distribution board 1, according to a first embodiment, shown in FIG. 1, comprises an electrical unit 3, which will be detailed below, and a plate 7 to which the electrical unit 3 is attached. The plate 7 forms at least part of the rear panel of the table 1.

For reasons that will become apparent later, the plate 7 is advantageously made from a thermally conductive material, preferably metal.

The electrical unit 3 is preferably, but not exclusively, a circuit breaker, in this case a three-phase circuit breaker. This circuit-breaker allows an electric current, in a manner known per se, to pass through an electrical installation and cuts off the electric current in the event of a short-circuit or overcurrent in the electrical installation.

In order to supply the electrical unit 3 with power or to pass an electrical current through it, in this case a three-phase current, the distribution board 1 comprises one or more electrically conductive members 4 which connect the electrical unit 3 to an electrical network, for example a power supply network. When the aforementioned network is three-phase, as in the example considered here, the electrically conductive members 4 include three phase electrically conductive members, which are respectively referenced 4A, 4B and 4C in FIG. 1, and one neutral electrically conductive member, referenced 4D. In practice, the electrically conductive members 4A, 4B, 4C and 4D are cables, as shown in FIGS. 1, 2, 3 and 4, or flexible or rigid bars, commonly referred to as “busbars”.

The electrically conductive member 4A is described in more detail below, it being understood that this description is applicable to the other electrically conductive members 4B, 4C and 4D. As can be seen in FIGS. 2 to 4, the electrical conductor 4A comprises a terminal 41A at the end connecting it to the electrical unit 3. This terminal 41A comprises two main faces S41A and S′41A, which are parallel to each other and opposite each other along a geometric axis, noted Z in FIGS. 2 to 4, in which this axis extends vertically, it being understood that this spatial orientation is not restrictive. A hole T41A passes right through the terminal 41A along the Z axis.

Advantageously, the two main faces S41A and S′41A of the terminal 41A are flat and normal to the Z axis.

Returning to the description of the electrical unit 3, the latter comprises a housing 5 which forms an electrically insulating enclosure inside which components of the electrical unit 3 are arranged, providing a main electrical function of the electrical unit 3, such as an electrical transit function and a short-circuiting function in the case where the electrical unit 3 is a circuit breaker.

The housing 5 is made of an electrically insulating material, typically a plastic, in particular a moulded plastic.

The electrical unit 3 also has one or more terminal blocks 6, of which there are four here because the electrical unit 3 is three-phase. The terminal blocks 6 allow the electrical unit 3 to be connected to the conductive members 4, thus forming an electrical input or output for the electrical unit 3, through which the current flows to reach the aforementioned components which, inside the housing 5, perform the main electrical function of the electrical unit 3. To this end, the terminal blocks 6 are made of an electrically conductive material, preferably metal.

The terminal blocks 6 are respectively associated with the electrically conductive members 4A to 4D in such a way that each of the terminal blocks 6 and the electrically conductive member associated with the terminal block 6 in question are electrically connected to each other by a connection device 2, described in detail below. In FIG. 1, 2A is the connection device between the electrically conductive member 4A and the associated terminal block 6, the latter being visible in FIGS. 2 to 4 and referred to therein as 6A; 2B is the connection device between the electrically conductive member 4B and the associated terminal block 6; 2C is the connection device between the electrically conductive member 4C and the associated terminal block 6; 2D is the connection device between the electrically conductive member 4D and the associated terminal block 6. In practice, the connection devices 2A, 2B, 2C and 2D are similar or even identical to each other, so that, below and with reference to FIGS. 2 to 4, only connection device 2A will be described in more detail, this description being applicable to the other connection devices 2B, 2C and 2D.

The terminal block 6A is described in more detail, on the understanding that in practice this description is applicable to the other three terminal blocks. As can be clearly seen from FIGS. 2, 3 and 4, the terminal block 6A comprises an end 61A having two main faces S61A and S′61A, which are parallel to each other and which are opposite each other along a geometrical axis, which, in use, i.e. when the terminal block 6A is connected to the conductive member 4A by the connection device 2A, is coincident, ignoring clearances, with the Z axis and which will therefore be considered to be the Z axis hereafter for convenience. A hole T61A passes right through the terminal 61A along the Z axis.

Advantageously, the two main faces S61A and S′61A of the terminal block 6A are flat and normal to the Z axis.

When the main face S61A of the terminal block 6A is in contact with the main face S′41A of the electrically conductive member 4A, the electrical connection is made by contact, in this case flat contact, between the terminal block 6A and the electrically conductive member 4A.

As can be seen in FIGS. 2, 3 and 4, the connection device 2A comprises a screw 21A, a lock washer 22A, a nut 23A, a core 25A and a support 27A.

The screw 21A is used to tighten the connection device 2A. To this end, the screw 21A comprises a threaded shank 212A and a head 214A, which are centred on a geometric axis, which, in use, is coincident, ignoring clearances, with the Z axis and which will therefore be considered to be the Z axis hereafter for convenience. Advantageously, the screw 21A is made of metal.

The lock washer 22A distributes the clamping forces applied by the screw 21A. The lock washer 22A is designed to be fitted coaxially around the threaded spindle 212A when in use. Advantageously, the lock washer 22A is made of metal.

The nut 23A enables the screw 21A to be screwed into the connection device 2A. The nut 23A comprises a first face S23A and a second face S′23A, which are parallel to each other and which are opposite each other along a geometric axis, which, in use, is coincident, ignoring clearances, with the Z axis and which will therefore be considered to be the Z axis hereafter for convenience. The first and second faces S23A and S′23A are joined together by a side face S″23 of the nut. A threaded hole T23A connecting the faces S23A and S′23A passes through the nut 23A along the Z axis.

The nut 23A is made of a thermally conductive material, preferably metal.

Advantageously, the nut 23A is parallelepipedic in shape and its two faces S23A and S′23A are flat and normal to the Z axis. Advantageously, the internally threaded hole T23A is centred on faces S23A and S′23A.

When the first face S23A of the nut 23A is in contact with the main face S′61A of the terminal block 6A, the thermal connection is ensured by contact, in this case flat contact, between the nut 23A and the terminal block 6A.

The core 25A transmits heat by thermal conduction within the connection device 2A. The core 25A, which is distinct from the nut 23A, is provided with two axial end faces S25A and S′25A, which are parallel to each other and which are opposite each other along a geometric axis, which, in service, is coincident, ignoring clearances, with the Z axis and which will therefore be considered to be the Z axis hereafter for convenience.

The core 25A is made of an electrically insulating, thermally conductive material, preferably ceramic.

Advantageously, the axial end faces S25A and S′25A are flat and normal to the Z axis.

In use, the axial end face S25A of the core 25A is designed to be in contact with the second face S′23A of the nut 23A, ensuring heat conduction by contact, in this case flat contact, between the core 25A and the nut 23A. Also in use, the axial end face S′25A of the core 25A is designed to be in contact with the plate 7, ensuring heat conduction by contact, in this case flat contact, between the core 25A and the plate 7.

Furthermore, the axial end faces S25A and S′25A are connected to each other by a side face S″25A of the core 25A. Advantageously, this side face S″25 has a dimension along the Z axis which is much greater than the corresponding axial dimension of the nut, in particular greater than twice or even three times the corresponding axial dimension of the nut. Also advantageously, the side face S″25 is generally cylindrical, being substantially centred on the Z axis and having a cross-section with a substantially rectangular profile with rounded corners.

In addition, the core 25A advantageously delimits an internal volume V25A, which opens onto the axial end face S25A, being substantially centred on the Z axis, and which is dimensioned to partially receive the shank 212A of the screw 21A. Thus, in the assembled state of the connection device 2A, the internal volume V25 opens into the internally threaded hole T23A of the nut 23A so that the screw 21A is partially received in the internal volume V25A when it is screwed into the internally threaded hole T23A.

The support 27A supports the nut 23A and the core 25A so as both to hold the nut 23A firmly in position and to transfer heat by conduction between the core 25A and the support 27A.

To this end, the support 27A comprises an internal volume V27A which is centred on a geometric axis, which, in use, is coincident, ignoring clearances, with the Z axis and which will therefore be considered to be the Z axis hereafter for convenience. The internal volume 27A delimits an internal face S27A which is adjusted in a complementary manner to the side face S″25A of the core 25, advantageously all around the Z axis. Thus, when the core 25A is received in the internal volume V27A of the support 27A, the shape of the internal face S27A of the support 27A coincides with the shape of the side face S″25A so that the support 27A covers most, or even almost all, of the side face S″25 so that there is maximum contact between the internal wall S27A and the side face S″25A, in particular with no air gap between them.

Here, the substantially rectangular cross-section of the side face S″25A means that, due to their complementary shapes with the inner face 27A of the support 27A, the core 25A and the support 27A are, in the assembled state, rotationally locked about the Z axis relative to each other. In order to retain the core 25A and the support 27 at least temporarily relative to one another along the Z axis, the core 23A and the support 27 are provided here with ad hoc retaining means, including at least one external protuberance 251A of the core 25A and an aperture 271A of the support 27A, which cooperate with one another by complementary shapes. Of course, other embodiments for the above-mentioned retaining means are conceivable.

At one of its axial ends, which, in use, faces the nut 23A, the support 27A is provided with protrusions 272A which are designed, in particular by their shape and size, to rotationally lock the nut 23A about the Z axis in the assembled state of the connection device 2A. In particular, when the nut 23A is positioned on the support 27A, the protrusions 272A coincide with the shape of the side face S″23A of the nut 25A and, by contact with the side face S″23 of the nut 23A, rotationally lock the nut 23A about the Z axis. The specific features of these protrusions 272A are not limiting, it being understood that they take advantage of the parallelepiped shape of the nut 23A.

More generally, the support 27A is provided with features that enable both the nut 23A and the core 25A to be held in position relative to it and a substantial contact interface, typically cylindrical centred on the Z axis, to be formed between it and the core 25A for the purposes of heat conduction between them. In practice, there are many possible embodiments for these fittings, other than the internal volume 27A, the aforementioned retaining means and the protrusions 272A, which have been detailed above by way of example. In an advantageous variant, not shown in the figures, the support 27A is overmoulded onto the core 25A and/or the nut 23A.

In any case, in the embodiment illustrated in FIGS. 1 to 4, the core 25A and the support 27A are designed so that, in the assembled state of the connection device 2A, leave the end face S′25A of the core to be arranged outside the core 27A, as clearly visible in FIG. 2. The face S′25A of the core 25 thus forms a free end of the core, which emerges from support 27A.

In addition, the support 27A has another function within the connection device 2A, namely to enable it to be affixed to the housing 5 of the electrical unit 3 and thus to enable the connection device 2 to be assembled to this housing 5.

To this end, the support 27A comprises external grooves 279A adapted to receive, advantageously by sliding in their longitudinal direction, complementary rails 51 of the housing 5, in particular provided to evacuate gases from an electrical cut-out chamber located inside the housing 5 in the case where the electrical unit 3 is a circuit breaker.

Of course, many other designs are possible for the external fittings of the support 27A, which enable it to be affixed to the housing 5.

In all cases, the support 27A is made of a material that emits a release containing microparticles and/or at least one gaseous compound when heated above a predetermined temperature threshold. By way of example, this material is a thermoplastic polymer, for example polyvinyl chloride, also known as PVC, or polyamide 6/6, polyethylene terephthalate, also known as PET, or polybutylene terephthalate, also known as PBT.

Advantageously, this support material 27A is electrically insulating.

In order to connect the electrically conductive member 4A and the terminal block 6A, the screw 2A is arranged along the Z axis successively through the washer 22A, the electrically conductive member 4A, and the terminal block 6A and is tightened in the nut 23A, being partially housed in the internal volume V25A of the core 25A. In this way, the electrically conductive member 4A and the terminal block 6A are pressed between the washer 22A and the nut 23A. The contact between the electrically conductive member 4A and the terminal block 6A ensures a good electrical connection. The electrically insulating nature of the respective materials of the housing 5 and core 2A means that the connection is electrically isolated from the outside, avoiding electrical risks to a user.

In the event of incorrect tightening, contact between the electrically conductive member 4A and the terminal block 6A is only partially ensured and the air in the space between the electrically conductive member 4A and the terminal block 6A creates an electrical resistance which generates heat when an electric current is passed through it. The heating is transmitted from the terminal block 6A to the nut 23A by conduction through the contact interface between the first face S23A of the nut 23A and the main face S′61A of the terminal block 6A. The thermal phenomenon is facilitated by the thermally conductive nature of the materials of the terminal block 6A and the nut 23A and by the flat contact between the terminal block 6A and the nut 23A.

The heating is then transmitted from the nut 23A to the core 25A by conduction through the contact interface between the axial end face S25A of the core 25A and the second face S′23A of the nut 23A. The thermal phenomenon is facilitated by the thermally conductive nature of the materials of the nut 23A and the core 25A and by the flat contact between the nut 23A and the core 25A.

The heating is then transmitted from the core 25A to the support 27A by conduction through the contact interface between the internal face S27A of the support 27A and the side face S″25 of the core. The thermal phenomenon is facilitated by the thermally conductive nature of the material of the core 25A and by the large contact interface provided by the enveloping of the side face S″25A of the core 25A by the internal face S27A of the support 27A.

When heated to a temperature above the aforementioned predetermined temperature threshold, the support 27A emits a release containing microparticles and/or at least one gaseous compound. When the unit is operating at full power, the release is immediate in the event of incorrect tightening, thanks to the rapidity of the physical effect of heat conduction. A detector 9 for gases, microparticles and/or volatile organic compounds, as described in EP3512056B1, is advantageously included in the distribution board 1 and its alarm is triggered when the release is emitted.

The flat contact between the plate 7 and the face S′25A of the core 25A advantageously enables the electrical unit 3 to be cooled by dissipating heat by thermal conduction and prevents overheating and damage to the electrical unit 3.

In a variant not shown, the position of the electrically conductive member 4 and of the terminal block is reversed, i.e. the main face S41A of the conducting member 4A is in contact with the main face S′61A of the terminal block 6A and that the first face S23A of the nut 23A is in contact with the main face S′41A of the electrically conducting member S″41. The operation of the electrical unit 3 is identical, but this variant allows the user to position the electrically conductive member 4A and the terminal block 6A in the most convenient order for making the connection.

FIG. 5 illustrates a second embodiment of the connection device 2A, identical to the first embodiment except for the following features. The support 27A has a bottom wall 277A which extends transversely to the Z axis, closing off the internal volume V27A. The core 25A does not emerge from the support 27A and its face S′25A is covered by an internal face S277A of the bottom wall 277A. In operation, the bottom wall is interposed, along the Z axis, between the core 25A and the plate 7.

In this second embodiment, the core 25A and the nut 23 can therefore be electrically conductive, particularly made of metal, without risk to the user. As an alternative to this second embodiment, not shown, the core 25A is advantageously integral with the nut 23A from the second face S′23A of the nut 23A. In other words, the nut 23A and core 25A are in one piece.

It should also be noted that the screw 21A and the lock washer 22A may be pre-existing parts belonging to the electrical unit 3 in a prior art connection device, which may also be replaced by the connection device 2.

Any feature described above for one embodiment or variant is applicable to the other embodiments and variants described above, insofar as this is technically possible.

1. An electrical connection device between an electrically conductive member and a terminal block of an electrical unit, the connection device comprising:
a nut, thermally conductive and provided with first and second faces opposite each other along an axis, the first and second faces being connected to each other by an internal thread of the nut, which extends along the axis and which is adapted to receive a screw that, by screwing into the internal thread, clamps together the electrically conductive member and the terminal block, by pressing them against the first face of the nut,
a core, which is thermally conductive and is bonded to the second face of the nut so as to transfer heat by conduction between the nut and the core, and
a support, which is adapted to be affixed to an electrically insulating housing of the electrical unit and which supports the nut and the core so as both to hold the nut securely in position and to transfer heat by conduction between the core and the support, the support being made of a material that emits a release containing microparticles and/or at least one gaseous compound when this material is heated above a predetermined temperature threshold.

2. The connection device according to claim 1, wherein the first face of the nut is flat and extends perpendicularly to the axis.

3. The connection device according to claim 1, wherein the nut and the core are two separate parts, and wherein the core is provided with a axial end face, which extends transversely to the axis and which arranged in axial contact against the second face of the nut.

4. The connection device according to claim 3, wherein the second face of the nut and the axial end face of the core are flat and extend perpendicularly to the axis.

5. The connection device according to claim 3, wherein the core is electrically insulating.

6. The connection device according to claim 5; wherein the core is made of ceramic.

7. The connection device according to claim 5, wherein the core is provided with a free end, which is opposite along the axis to the axial end face and which emerges from the support.

8. The connection device according to claim 1, wherein the core is integral with the nut from the second face of the nut, and wherein the nut and the core are electrically conductive.

9. The connection device according to claim 8, wherein the nut and the core are made of metal.

10. The connection device according to claim 1, wherein the core is provided with a side face, which is cylindrical overall, being substantially centred on the axis, and which is at least partially covered in contact by the support.

11. An electrical unit, for example a switching device, comprising:
an electrically insulating housing,
at least one terminal block, which forms an electrical input or an electrical output of the electrical unit, and
for the or each terminal block, a connection device between an electrically conductive member and the terminal block, the device being according to claim 1.

12. A distribution board comprising:
an electrical unit according to claim 11, and
for the or each terminal block, an electrically conductive member which is connected to the terminal block by the connection device.

13. A distribution board comprising:
an electrical unit comprising:
an electrically insulating housing,
at least one terminal block, which forms an electrical input or an electrical output of the electrical unit, and
for the or each terminal block, an electrically conductive member which is connected to the terminal block by the connection device according to claim 7, and
a plate to which the electrical unit is attached, the plate being thermally conductive and being in contact with the free end of the core of the connection device so as to transfer heat by thermal conduction between the core and the plate.
